(19) United States
(12) Patent Application Publication
(10) Pub. No.: US 2006/0199240 A1
(43) Pub. Date: Sep. 7, 2006

(54) Biosensor and method comprising enzymes immobilized on semiconductors

(75) Inventors: Itarmar Willner, Zion (IL); Eugenii Katz, Jerusalem (IL); Vered Yissar, Neve-Monoson (IL)

Correspondence Address: BROWDY AND NEIMARK, P.L.L.C.;624 NINTH STREET, NW, SUITE 300, WASHINGTON, DC, 20001-5303, US

(73) Assignee: Yissum Research Development Company of the Hebrew University of Jerusalem, jerusalem (IL)

(21) Appl. No.: 10/536,032

(22) PCT Filed: Oct. 2, 2003
(86) PCT No.: PCT/IL03/00795

§ 371 Date: Mar. 6, 2006

(30) Foreign Application Priority Data
Nov. 21, 2002 (IL) 153012

(51) Int. Cl. C12Q 1/54; C12Q 1/26; C12M 1/34

(52) U.S. Cl. 435/14; 435/25; 435/287.1

(57) ABSTRACT

The present invention provides a method and a device that utilizes functionalized semiconductor element for detecting presence and/or concentration of an agent in an assayed sample. The device of the present invention comprises: (i) a body having a surface comprising or having associated thereto semi-conducting material that can be excited such that in the presence of an electron donor, said semi-conducting material can generate an electric current within the body; and (ii) an enzyme attached to said semi-conducting material which in the presence of a substrate said enzyme catalyzes a reaction that yields said electron donors.

### FIELD OF THE INVENTION

This invention relates to an analytical method for the determination of the presence and/or concentration of an analyte in a liquid medium. The method of the present invention is a photoelectrochemical method in which the concentration or the presence of the analyte is determined by means of measurements of a current or voltage, the formation of which is dependent on an enzymatic reaction.

### LIST OF REFERENCES

The following references are considered to be pertinent for the purpose of understanding the background of the present invention.
1. Klein, D. L.; Roth, R.; Lim, A. K. L.; Alivisatos, A. P.; McEuen, P. L., Nature 1997, 389, 699-701.
2. Alivisatos, A. P., Science 1996, 271, 933-937.
3. Kim, T. W.; Lee, D. U.; Yoon, Y. S., J. Appl. Phys. 2000, 88, 3759-3761.
4. Bruchez, M., Jr.; Moronne, M.; Gin, P.; Weiss, S.; Alivisatos, A. P., Science 1998, 281, 2013-2015.
5. Chan, W. C. W.; Nie, S., Science 1998, 281, 2016-2018.
6. Willner, I.; Patolsky, F.; Wasserman, J., Angew. Chem. Int. Ed. 2001, 40, 1861-1864
7. Tessler, N.; Medvedev, V.; Kazes, M.; Kan, S.; Banin, U., Science 2002, 295, 1506-1508.
8. Pavesi, L.; Negro, L. D.; Mazzoleni, C.; Franzo, G.; Priolo, F., Nature 2000, 408, 440-444.
9. Malko, A. V.; Mikhailovsky, A. A.; Petruska, M. A.; Hollingsworth, J. A.; Htoon, H.; Bawendi, M. G.; Klimov, V. I., Appl. Phys. Lett. 2002, 81, 1303-1305.
10. Gerion, D.; Parak, W. J.; Williams, S. C.; Zanchet, D.; Micheel, C. M.; Alivisatos, A. P., J. Am. Chem. Soc. 2002, 124, 7070-7074.
11. Pathak, S.; Choi, S.-K.; Arnheim, N.; Thompson, M. E., J. Am. Chem. Soc. 2001, 123, 4103-4104.
12. Reynholds, III, R. A.; Mirkin, C. A.; Letsinger, R. L., J. Am. Chem. Soc. 2000, 122, 3795-3796.
13. Niemeyer, C. M., Angew. Chem. Int. Ed. 2001, 40, 4128-4158.

The above publications will be referenced bellow by indicating their number from the above list.

### BACKGROUND OF THE INVENTION

The unique electronic and photonic properties of semiconductor quantum dots have been used in a range of optoelectronic applications.^{1,2} Specifically, the photophysical features of semiconductor nanoparticles are employed to develop sensor³ and biosensor systems,⁴⁻⁶ light emitting diodes⁷ and lasers.^{8,9} Protein functionalized quantum-size semiconductor particles or antibody-modified nanoparticles were suggested as luminescent labels for biorecognition events.¹⁰ Similarly, nucleic acid modified semiconductor nanoparticles were reported to act as luminescent probes for DNA hybridization.^{6,11} Recently, oligonucleotide derivatized quantum dots were used as building blocks to form extended networks of DNA crosslinked nanoparticles, and the photoelectrochemical features of the arrays were examined.^{12,13 SUMMARY OF THE INVENTION}

The present invention provides a method and a device that utilizes functionalized semiconductor element, typically in the form of particles, preferably semiconductor nanoparticles, for detecting presence and/or concentration of an agent in an assayed sample. The semiconductor element has attached thereto an enzyme, which in the presence of a substrate catalyzes a reaction, yielding a product that acts as an electron donor for the holes generated in the valence-band of the semiconductor body by excitation. The analyte is such that it affects the ability of the enzyme to cause generation of the electron donors or the analyte is one of the reactants in the reaction that produces electron donors.

Thus, according to a first aspect, the present invention provides a device comprising:
(a) a body having a surface made of or having associated thereto semi-conducting material that can be excited such that in the presence of an electron donor, said semi-conducting material can generate an electric current within the body; and
(b) an enzyme attached to said semi-conducting material which in the presence of a substrate said enzyme catalyzes a reaction that yields said electron donors.

The device is typically used for assaying an analyte in a sample. In this embodiment, the analyte may be the enzyme's substrate, or may be a modulator of the enzymes activity, e.g. an inhibitor, a co-factor, etc. In the presence of the analyte the electric current may be generated or modulated. This may provide an indication for the presence of the analyte in the assayed sample. The level of the electric current or the extent of the modulation of the electric current may serve as an indication of the concentration of the analyte in the assayed sample. The term “determination” will be used herein to denote both qualitative assaying of the analyte, namely to get a Yes/No answer whether the analyte exists in the assayed sample, as well as a quantitative assaying, namely determine the presence as well as the concentration of the analyte in the sample.

According to a preferred embodiment, the body is an electrode having associated thereto a layer comprising particles made of a semiconducting material, more preferably nanoparticles made of such material. According to another preferred embodiment, the electrode itself is made of or is coated by a semiconducting material. A hybrid system is formed between the semiconducting material and an enzyme, such that upon excitation, e.g. through irradiation with electromagnetic radiation, and in the presence of electron donor, an electric current is generated.

The flow of current is an electric response that results from a reaction occurring in the assayed sample that generates electron donors. The formation of the electron donors is affected by the presence of the analyte in the assayed sample or the analyte itself may be one of the reactants in the reaction. The term “electric response” refers to any measurable change in the electrical parameters recorded by or electrical properties of the electrode. An electric response may be flow of current, charge or potential change that results from the reaction. As will no doubt be appreciated, the invention is not limited by the manner in which the electric response is measured and any manner of measurement that may be used therefor can be applied for measurement of the electric response.

The invention permits the qualitative detection of the presence of an analyte in an assayed sample by monitoring the electric response. In addition, by measuring the extent of the response, the concentration of the analyte may also be quantitatively determined.

Examples of enzymes are acetylcholine esterase (AChE), glucose oxidase, lactate dehydrogenase (LDH), fructose dehydrogenase, alcohol dehydrogenase, malate dehydrogenase, choline oxidase, etc. The electron donor may for example be the reaction product between the enzyme and the substrate, or may be generated from a cofactor of the enzyme. Preferably, the cofactor is either attached through a linker to the semi-conducting material or is solubilized in the assayed sample.

Electrodes in the device of the invention are made of or coated with conducting or semi-conducting materials, for example gold, platinum, palladium, silver, carbon, etc. Semi-conducting materials used in the present invention may be selected, for example, from Group III-V, Group III-V alloys, Group II-VI, Group I-VII, and Group IV semiconductors. Examples of Group III-V semiconductors are InAs, GaAs, GaP, GaSb, InP, InSb, AlAs, AlP, AlSb and alloys such as InGaAs, GaAsP, InAsP. Examples of Group II-VI semiconductors are CdS, CdSe, CdTe, ZnS, ZnSe, ZnTe, HgS, HgSe, HgTe and the like. Examples of Group I-VII semiconductors are CuCl, CuBr, CuI, AgCl, AgBr, AgI and the like. Examples of Group IV semiconductors are Si and Ge.

The excitation with electromagnetic radiation may be carried out at diverse wavelengths, depending on the sort of semi-conducting material used and on its form, e.g. particles, nanoparticles, quantum dots, nanorods, etc. For example, in the case of CdS nanoparticles, the excitation energy is in the UV-visible range. The excitation energy may also be tuned by coating the semi-conducting material with a suitable dye.

According to another aspect, the present invention provides a bio-sensing system for determining an analyte in an assayed sample, the system comprising:
(i) an irradiation unit;
(ii) a reaction cell with a working electrode and a counterpart electrode, said working electrode having a surface comprising or having associated thereto semi-conducting material that generates current within the working electrode upon excitation with said irradiation unit and in the presence of an electron donor, and an enzyme attached to said semi-conducting material, such that in the presence of the enzyme's substrate said enzyme catalyzes a reaction that yields electron donors, said analyte being said substrate or a modulator that can modulate the enzyme's catalytic activity; and
(iii) measuring utility for reading the current or voltage generated within the working electrode.

Also provided by the present invention, a method for identifying the presence of an analyte in an assayed sample. The method comprises providing a bio-sensing system as defined above, introducing the sample to be assayed into the reaction cell of the system, irradiating the system so as to cause excitation of the semiconducting particles and measuring the electrical response, a change in the electrical response as compared to an electrical response under the same condition in a control medium which does not comprise the analyte, indicating the presence of the analyte in the system.

Also provided by the present invention, a method for measuring the concentration of an analyte in an assayed sample, comprising: providing a bio-sensing system as defined above, introducing the sample to be assayed into the reaction cell of the system, irradiating the system so as to cause excitation of the semi-conducting particles and measuring the electrical response, the magnitude of the electrical response as compared to a calibration curve of the electrical responses under the same conditions in mediums which comprise known concentrations of the analyte, indicating the concentration of the analyte in the system.

According to another aspect, the present invention further provides a bio-sensing system for determining the presence of one or more different analytes in an assayed sample, the system comprising:
(i) an irradiation unit
(ii) a reaction cell with an array of bio-sensing systems each comprising:
   a. a working electrode and a counterpart electrode, said working electrode having a surface comprising or having associated thereto semi-conducting material that generates current within the working electrode upon excitation with said irradiation unit and in the presence of an electron donor, and an enzyme attached to said semi-conducting material, such that in the presence of the enzyme's substrate said enzyme catalyzes a reaction that yields electron donors, said analyte being said substrate or a modulator that can modulate the enzyme's catalytic activity; and
   b. measuring utility for reading the current or voltage generated within each of the working electrodes.

### BRIEF DESCRIPTION OF THE DRAWINGS

In order to understand the invention and to see how it may be carried out in practice, some preferred embodiments will now be described, by way of non-limiting examples only, with reference to the accompanying drawings, in which:

FIG. 1 illustrates the assembly of the CdS-nanoparticle/acetylcholine esterase (AchE) hybrid system for the photoelectrochemical assay of ACHE activity and the generation of a photoelectric current in the presence of acetylthiocholine.

FIG. 2A is a graph showing the photocurrent spectra in the presence of variable concentrations of acetylthiocholine

FIG. 2B shows a calibration curve corresponding to the photocurrent at λ=380 nm at variable concentrations of acetylthiocholine

FIG. 3A shows the photocurrent spectra of the AChE-functionalized CdS-nanoparticle electrode in the presence of 10 mM acetylthiocholine and different concentrations of the inhibitor 1,5-bis(4-allydimethylammoniumphenyl)pentane-3-one dibromide. Inset: Lineweaver-Burke plots corresponding to the photocurrent at variable concentrations of acetylthiocholine (1), in the presence of: (a) 0 μM of the inhibitor (3); (b) 10 μM of (3); (c) 20 μM of (3). Data were recorded in 0.1 M phosphate buffer, pH=8.1, under argon.

FIG. 3B illustrates the effect of the inhibitor on the current generated in the presence of acetylthiocholine by a monolayer consisting of a CdS-nanoparticle/AChE hybrid system associated with an electrode.

FIG. 3C shows Lineweaver-Burke plots corresponding to the photocurrent at variable concentrations of acetylthiocholine, in the presence of: (a) 0 mM of acetylthiocholine; (b) 1 mM acetylthiocholine; (c) 2 mM acetylthiocholine. Data were recorded in 0.1 M phosphate buffer, pH=8.1, under argon.

FIG. 4A illustrates the assembly of the CdS-nanoparticle/Lactate dehydrogenase (LDH) system, where the cofactor NAD⁺ is solubilized in the assayed sample.

FIG. 4B illustrates the assembly of the CdS-nanoparticle/LDH system for the photoelectrochemical detection of lactate, where the cofactor NAD⁺ is covalently immobilized to the system.

FIG. 5A illustrates the preparation of the system shown in FIG. 4A.

FIG. 5B illustrates the preparation of a dimer of two systems shown in FIG. 4B.

### DETAILED DESCRIPTION OF THE INVENTION

Acetylcholine esterase inhibitors and activators may be detected photoelectrochemically according to the present invention, in a bio-sensing system comprising acetylcholine esterase (AChE) attached covalently or physically to nanoparticles of semi-conducting material such as CdS.

Acetylcholine (ACh) is a central neurotransmitter that activates the synapse and the neural response. The neurotransmitter, ACh, after activating the neural response, is rapidly hydrolyzed by the serine protease ACHE to restore the resting potential of the synaptic membrane. Different reagents, such as the nerve gas diisopropyl fluorophosphate (Sarin) or toxins (e.g. cobratoxin) act as inhibitors or blockers of AChE. Blocking of the enzyme-stimulated nerve conduction leads to rapid paralysis of vital functions of living systems. Thus, the assembly described here may be considered as a biomaterial-semiconductor hybrid device acting as biosensor for biological warfare nerve gases.

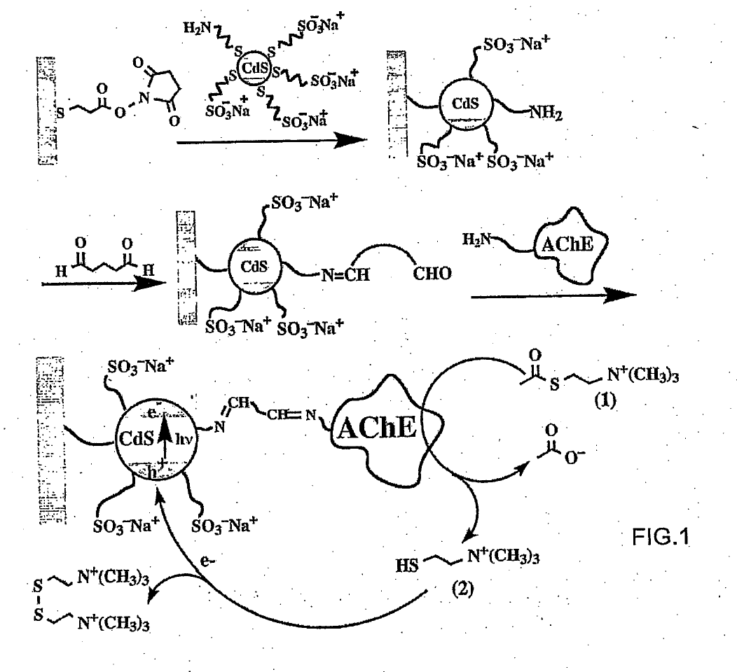

CdS nanoparticles (diameter 3 nm) were capped with a protecting monolayer of cysteamine and mercaptoethan sulfonic acid. XPS analysis indicates that ca. 84% of the Cd²⁺ surface groups are linked to the thiolated molecules and that the ratio between the cysteamine and thiol sulfonate units is ca. 1:10, respectively. The capped CdS nanoparticles were covalently linked to an Au-electrode functionalized with an N-hydroxysuccinimide active ester cysteic acid, as shown in FIG. 1. Microgravimetric quartz crystal microbalance (QCM) measurements for the analogous association of the CdS nanoparticles on an Au-quartz crystal, indicate that the binding of the CdS nanoparticles to the surface involves a change of Δf=140 Hz that corresponds to a surface coverage of 5.7×10¹² particles·cm⁻². The AChE was then covalently linked to the CdS nanoparticles using glutaric dialdehyde as bridging unit. Parallel microgravimetric QCM measurements indicate that the surface coverage of AChE is 3.9×10⁻¹² mole·cm⁻². Thus ca. 2.4 nanoparticles are associated with each AChE unit.

As depicted in FIG. 1, the CdS nanoparticle-AChE hybrid system is photoelectrochemically active in the presence of acetylthiocholine, (1), as substrate. One of the products of the hydrolysis of acetylthiocholine is thiocholine (2), which is an electron donor.

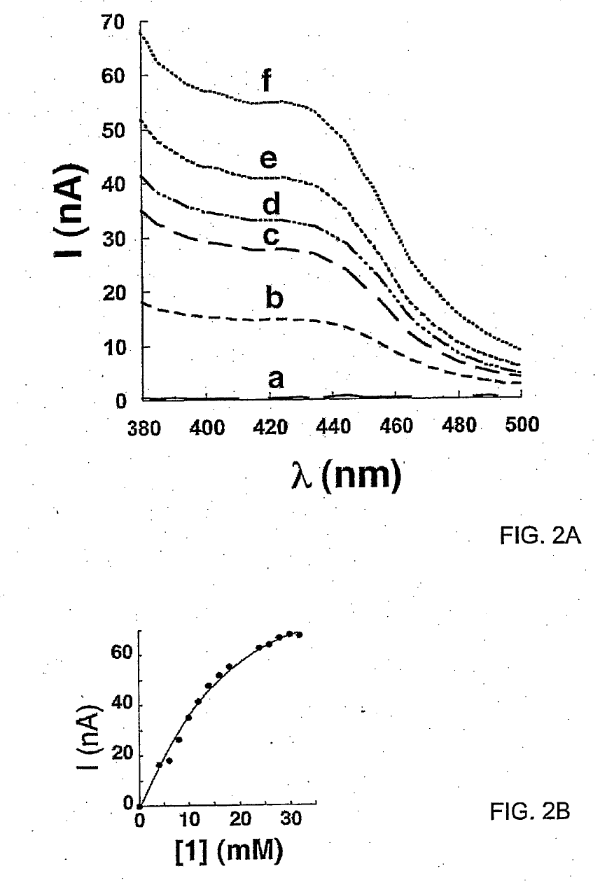

FIG. 2A depicts the photocurrent action spectra resulting in the photoirradiation of the system in the presence of different concentrations of acetylthiocholine. The photocurrent spectra overlap the absorption spectrum of the CdS nanoparticles, implying that the photocurrent originates from the excitation of the semi-conducting nanoparticles. Control experiments reveal that no photocurrent is generated in the system in the absence of acetylthiocholine. Also, irradiation of the CdS nanoparticle monolayer that lacks AChE in the presence of acetylthiocholine does not yield any photocurrent. Thus, the photocurrent generation in the system is attributed to the AChE catalyzed hydrolysis of acetylthiocholine, (1), to acetate and thiocholine, (2). The latter product acts as donor for the holes generated in the valence band upon excitation of the CdS nanoparticles. Thus, oxidation of thiocholine by the holes eliminates the electron-hole recombination, and thus a steady-state photocurrent is generated. As the concentration of (1) is elevated, the concentration of (2) at the particle surface is higher, and the photocurrent is enhanced, as shown in FIG. 2B.

In further control experiments it was found that the photocurrents generated by the AChE-functionalized CdS monolayer in the presence of different concentrations of the related electron donor cysteamine are similar to the photocurrents generated by the analogous concentrations of acetylthiocholine (1). These results suggest that all of the substrate (1) at the CdS nanoparticle interface is transformed to (2) by the biocatalyzed process, and that the oxidation of (2) by the valence-band holes is efficient and prevents the diffusion of (2) to the bulk solution.

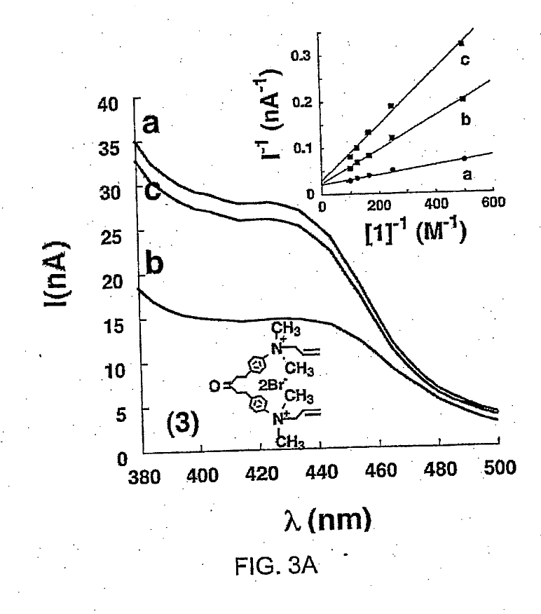

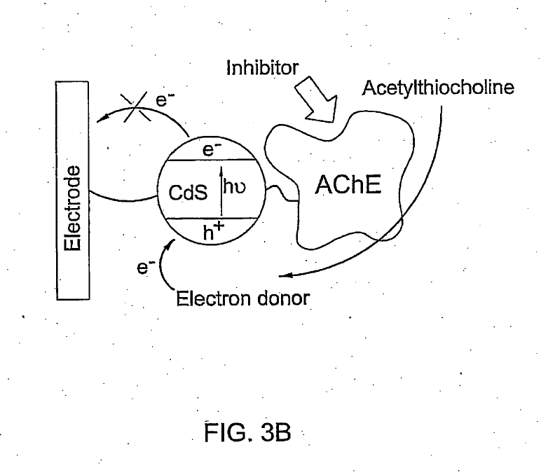

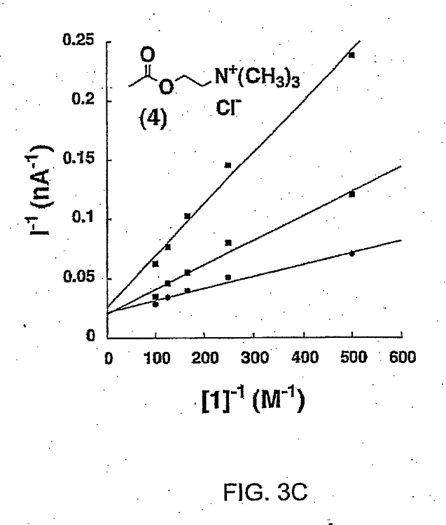

FIG. 3A shows the photocurrent action spectra of the AChE-functionalized CdS-nanoparticle electrode in the presence of 10 mM (1), and different concentrations of the inhibitor 1,5-bis(4-allyldimethylammoniumphenyl)pentane-3-one dibromide, (3). Increase of the concentration of (3) decreases the photocurrent. Washing off the inhibitor from the cell almost restores the initial photocurrent, as showed in curve (c).

FIG. 3A inset shows the Lineweaver-Burk plots that correspond to the inhibition of the photocurrents in the presence of different concentrations of (3). From these plots it may be concluded that (3) acts as competitive inhibitor K₁=7 μM. The K_{M} value of the AChE linked to the CdS nanoparticles towards acetylthiocholine, (1), is K_{M}=5 mM. This value is higher than the K_{M}=0.13 mM of ACHE and (1) in solution. The higher K_{M} value for the nanoparticle-immobilized ACHE may be attributed to slight deactivation and structural perturbation of the biocatalyst as a result of surface linkage.

The decrease in the observed photocurrent in the presence of the inhibitor is showed schematically in FIG. 3B and is attributed to the lower yields for the biocatalyzed formation of thiocholine, and thus less efficient removal of the valence-band holes. Related results are observed upon analyzing the photocurrents generated by the AChE-CdS nanoparticle/(1) system in the presence of different concentrations of the natural substrate of AchE, acetylcholine, (4), as shown in FIG. 3C. Acetylcholine, (4), competes with acetylthiocholine for the active sites. As a result, increase of acetylcholine results in a decrease in the observed photocurrent. The affinity of the enzyme to its natural substrate ACh is higher than its affinity to acetylthiocholine. Thus, the AChE-CdS nanoparticle/(1) system is highly sensitive to acetylcholine.

In the above example the driving force for the formation of the photocurrent is the biocatalyzed formation of thiocholine that scavenges the photogenerated valence-band holes. It was also demonstrated that enzyme inhibitors decrease the photocurrents, and thus the nanoparticle-AChE system acts as a biosensor for the respective inhibitor. Besides the immediate potential application of such biosensor for biological warfare, the CdS nanoparticle-AChE/acetylthiocholine system may be a versatile photoelectrochemical label for different biosensors.

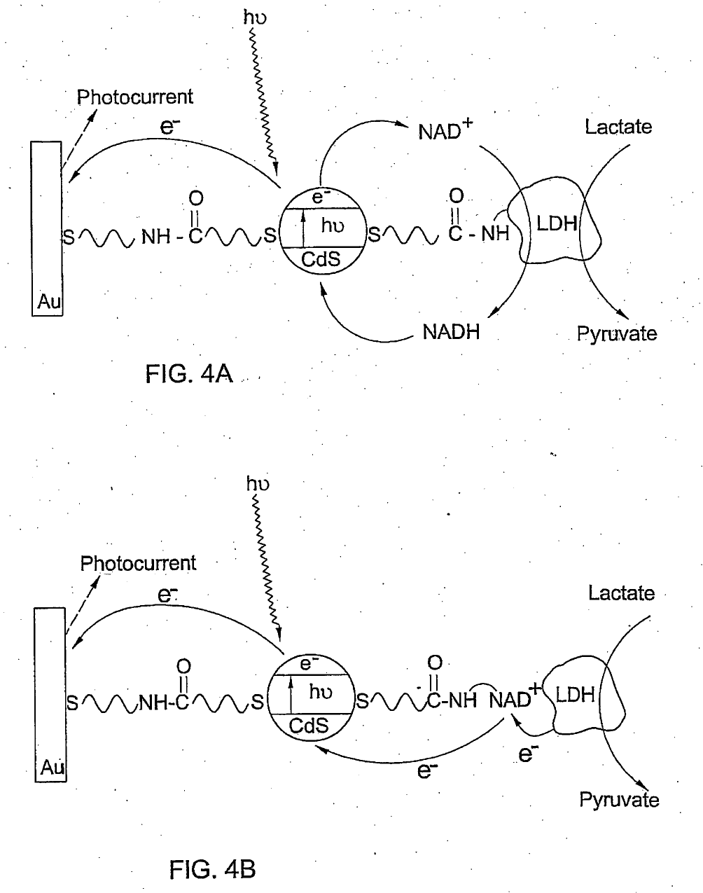

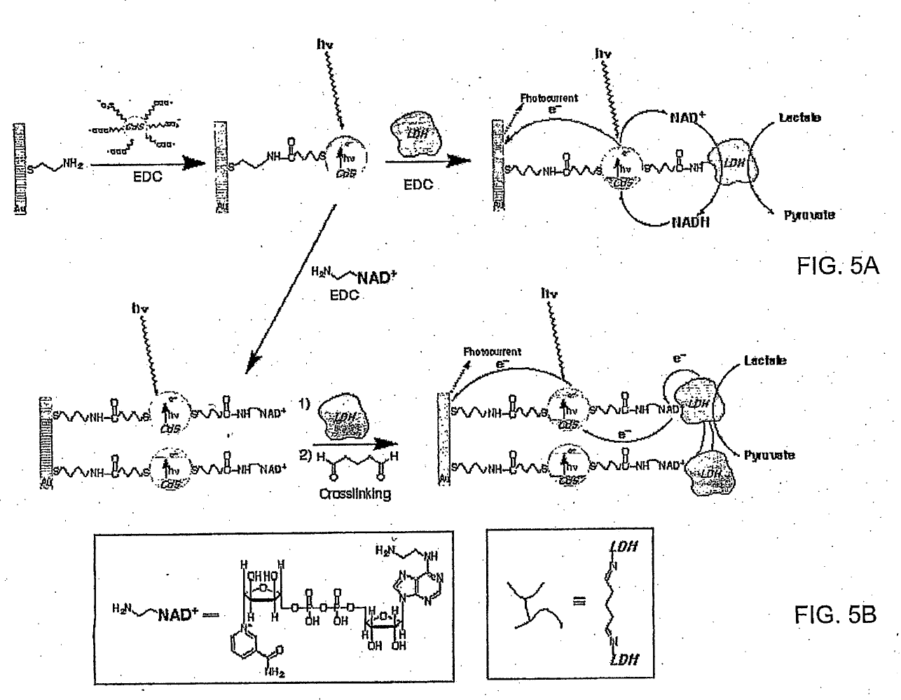

An additional example includes NAD(P)⁺ dependent enzymes connected to the CdS nanoparticles. In such examples, the cofactor can be solubilized or immobilized in the system. In the presence of the respective substrate, the enzyme reduces the NAD(P)⁺ cofactor yielding the respective reduced form NAD(P)H. The reduced cofactor can donate an electron to the CdS nanoparticles, thus maintaining a photocurrent upon the appropriate illumination. The photocurrent will be produced upon the following conditions: (a) CdS nanoparticles are co-immobilized with the NAD(P)⁺-dependent enzyme at the electrode surface, (b) the respective NAD(P)⁺ cofactor is added to the solution or co-immobilized in the system, (c) the respective enzyme substrate is added to the solution, (d) appropriate illumination is applied on the electrode surface. FIG. 4 outlines two possible configurations: (A) with the solubilized NAD⁺ cofactor, and (B) with the covalently immobilized NAD⁺ cofactor. In both cases the NAD⁺ dependent enzyme lactate dehydrogenase (LDH) has been used together with the respective substrate, lactate, which is biocatalytically oxidized to pyruvate. The enzymatic reaction results in the formation of the reduced cofactor NADH (solubilized in the part A and immobilized in the part B). The photocurrent value is proportional to the substrate (lactate) concentration: as long as the lactate concentration is below the enzyme saturating value, the light intensity is constant. FIG. 5A outlines the preparation of the system shown in FIG. 4A for the solubilized cofactor and FIG. 5B outlines the preparation of the system shown in FIG. 4B for the immobilized cofactor. The enzyme molecules in the example of FIG. 5B are cross-linked in a two-dimensional film with a cross-linker glutaric dialdehyde. This cross-linking is useful to stabilize the enzyme film and to prevent the enzyme desorption from the sensing interface.

1. A device comprising:
(i) a body having a surface comprising or having associated thereto semi-conducting material that can be excited such that in the presence of an electron donor, said semi-conducting material can generate an electric current within the body; and
(ii) an enzyme attached to said semi-conducting material which in the presence of a substrate said enzyme catalyzes a reaction that yields said electron donors.

2. The device of claim 1, wherein the body is made of or coated by a semi-conducting material.

3. The device of claim 1, wherein the body is an electrode having associated thereto particles comprising a semi-conducting material.

4. The device of claim 1, wherein the semi-conducting material is selected from: Group III-V, Group III-V alloys, Group II-VI, Group I-VII, and Group IV semiconductors.

5. The device of claim 1, wherein the semi-conducting material is selected from InAs, GaAs, GaP, GaSb, InP, InSb, AlAs, AlP, AlSb, InGaAs, GaAsP, InAsP, CdS, CdSe, CdTe, ZnS, ZnSe, ZnTe, HgS, HgSe, HgTe, CuCl, CuBr, CuI, AgCl, AgBr, AgI Si and Ge.

6. The device of claim 1, wherein the excitation of the semi-conducting material is by electromagnetic radiation.

7. The device of claim 6, wherein the electromagnetic radiation is in the UV or visible range.

8. The device of claim 1, wherein the semi-conducting material is coated with a dye.

9. The device of claim 1, wherein the enzyme is selected from acetylcholine esterase, glucose oxidase, lactate dehydrogenase, fructose dehydrogenase, alcohol dehydrogenase, malate dehydrogenase, and choline oxidase.

10. The device of claim 1, wherein the electron donor is the reaction product between the enzyme and the substrate.

11. The device of claim 1, wherein the electron donor is generated from a cofactor of the enzyme.

12. The device of claim 11, wherein the cofactor is selected from NAD⁺ and NADP⁺.

13. The device of claim 11, wherein the cofactor is attached through a linker to the semi-conducting material.

14. The device of claim 13, wherein the enzyme is attached to the semi-conducting material through the cofactor.

15. A bio-sensing system for determining an analyte in an assayed sample, the system comprising:
(i) an irradiation unit;
(ii) a reaction cell with a working electrode and a counterpart electrode, said working electrode having a surface comprising or having associated thereto semi-conducting material that generates current within the working electrode upon excitation with said irradiation unit, and in the presence of an electron donor, and an enzyme attached to said semi-conducting material, such that in the presence of the enzyme's substrate said enzyme catalyzes a reaction that yields electron donors, said analyte being said substrate or a modulator that can modulate the enzyme's catalytic activity; and
(iii) measuring utility for reading the current or voltage generated within the working electrode.

16. A method for identifying the presence of an analyte in an assayed sample, comprising: providing a bio-sensing system according to claim 15, introducing the sample to be assayed into the reaction cell of the system, irradiating the system so as to cause excitation of the semiconductor particles and measuring the electrical response, a change in the electrical response as compared to an electrical response under the same condition in a control medium which does not comprise the analyte, indicating the presence of the analyte in the system.

17. A method for measuring the concentration of an analyte in an assayed sample, comprising: providing a bio-sensing system according to claim 15, introducing the sample to be assayed into the reaction cell of the system, irradiating the system so as to cause excitation of the semiconductor particles and measuring the electrical response, the magnitude of the electrical response as compared to a calibration curve of the electrical responses under the same conditions in mediums which comprise known concentrations of the analyte, indicating the concentration of the analyte in the system.

18. A bio-sensing system for determining the presence of one or more different analytes in an assayed sample, the system comprising:
(i) an irradiation unit, and
(ii) a reaction cell with an array of bio-sensing systems each comprising:
a. a working electrode and a counterpart electrode, said working electrode having a surface comprising or having associated thereto semi-conducting material that generates current within the working electrode upon excitation with said irradiation unit and in the presence of an electron donor, and an enzyme attached to said semi-conducting material, such that in the presence of the enzyme's substrate said enzyme catalyzes a reaction that yields electron donors, said analyte being said substrate or a modulator that can modulate the enzyme's catalytic activity; and
b. measuring utility for reading the current or voltage generated within each of the working electrodes.
